(19) United States
(12) Patent Application Publication
(10) Pub. No.: US 2026/0175002 A1
(43) Pub. Date: Jun. 25, 2026

(54) METHOD AND APPARATUS FOR REDUCTION, REMOVAL, OR DISPERSION OF FILLER AGGREGATES

(71) Applicant: VIAS Partners, LLC, Doylestown, PA (US)

(72) Inventor: Sanjay Batra, New Hope, PA (US)

(73) Assignee: VIAS Partners, LLC, Doylestown, PA (US)

(21) Appl. No.: 19/426,532

(22) Filed: Dec. 19, 2025

Related U.S. Application Data
(60) Provisional application No. 63/737,981, filed on Dec. 23, 2024.

(51) Int. Cl. A61M 37/00; A61N 5/06

(52) U.S. Cl. CPC A61M 37/0092; A61N 5/0616; A61N 2005/0663

(57) ABSTRACT

The present disclosure provides a method for reducing, removing, or dispersing a dermal filler aggregate in a subject. The method includes contacting a target area of the subject’s skin with a device configured to apply vibrational or mechanical energy, and applying vibrational energy to the target area for a duration sufficient to facilitate reduction, removal, or dispersion of the dermal filler aggregate. The vibrational energy may be applied while the device is held stationary over the aggregate or moved across the target area in vertical, horizontal, circular, or sweeping motions. In certain embodiments, the device is configured to contact the skin, and visible light is optionally applied during treatment. Application of vibrational energy promotes mechanical disruption and redistribution of dermal filler material within the surrounding tissue, thereby reducing the palpability or appearance of filler nodules, clumps, or irregularities.

### CROSS-REFERENCE TO RELATED APPLICATIONS

This application claims priority to U.S. Application No. 63/737,981, titled METHOD AND APPARATUS FOR REDUCTION, REMOVAL, OR DISPERSION OF DERMAL FILLERAGGREGATES, filed December 23, 2024, which is hereby incorporated by reference in its entirety.

### FIELD OF INVENTION

The present disclosure relates to methods and apparatuses for dermatologic treatment, and more particularly to methods for reducing, removing, or dispersing dermal filler aggregates in a subject through the application of mechanical or vibrational energy to the skin.

### BACKGROUND

Dermal fillers are widely used in aesthetic and reconstructive procedures to restore volume, enhance facial contours, and reduce the appearance of wrinkles and fine lines. Following injection, however, dermal fillers may occasionally form aggregates, which can present as palpable or visible lumps, nodules, or irregularities beneath the skin. These aggregates may result from factors such as product distribution, injection technique, tissue characteristics, or post-procedural movement of the filler material. The presence of dermal filler aggregates can lead to aesthetic dissatisfaction, discomfort, or the need for corrective treatment.

Conventional approaches for managing dermal filler aggregates include manual massage, needle-based disruption, dissolution with enzymes such as hyaluronidase (for hyaluronic acid fillers), or, in more severe cases, surgical intervention. These methods can be uncomfortable, inconsistent in effectiveness, or limited to specific filler chemistries. In particular, fillers that are non-enzymatically degradable, such as calcium hydroxyapatite or polymethyl methacrylate, may be more challenging to correct using traditional dissolution techniques and can be permanent, unsightly, and disfiguring

There remains a need for improved, noninvasive methods that can effectively reduce, remove, or disperse dermal filler aggregates across a range of filler types. Such methods ideally would provide controlled mechanical action capable of redistributing filler within the tissue, improving aesthetic outcomes, and reducing the palpability or visibility of aggregates without requiring invasive procedures or chemical degradation of the filler material.

### SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the present disclosure are directed to methods for reducing, removing, or dispersing dermal filler aggregates in a subject. In certain embodiments, the method includes contacting a target area of the subject’s skin with a device configured to apply vibrational or mechanical energy, and applying vibrational energy to the target area for a duration sufficient to facilitate reduction, removal, or dispersion of the dermal filler aggregate. The vibrational energy can be delivered while the device is held stationary over the aggregate or moved across the target area using vertical, horizontal, circular, or sweeping motions.

Embodiments of the present disclosure can further include using a device having a contact surface with surface features such as non-penetrating tips configured to distribute vibrational energy across the skin. In certain embodiments, visible light may be applied during or after delivery of vibrational energy. The methods described herein can be performed on dermal fillers of various compositions, including hyaluronic acid, calcium hydroxyapatite, polymethyl methacrylate, and autologous or allogenic fat. The foregoing general description of the illustrative embodiments and the following detailed description thereof are merely exemplary aspects of the teachings of this disclosure and are not restrictive.

### BRIEF DESCRIPTION OF FIGURES

The features, and advantages of the invention will be apparent from the following more detailed description of certain embodiments of the invention and as illustrated in the accompanying drawings in which:

FIG. 1 illustrates a schematic representation of exemplary methods and apparatuses for reducing, removing, or dispersing a dermal filler aggregate in a subject, according to aspects of the present disclosure;

FIG. 2 illustrates a perspective view of a device with a body and a head, according to aspects of the present disclosure;

FIG. 3 illustrates a perspective view of the head of the device of FIG. 2, according to aspects of the present disclosure;

FIG. 4 illustrates a perspective view of the device of FIG. 2 and a cartridge configured for positioning within the body, according to aspects of the present disclosure;

FIG. 5 illustrates a flow diagram of a method for reducing, removing, or dispersing a dermal filler aggregate in a subject, according to aspects of the present disclosure;

FIG. 6 illustrates a graph demonstrating the effect of mechanical energy on the palpability and dispersal of dermal filler aggregates, according to aspects of the present disclosure;

FIG. 7A illustrates hydrogel images showing time-dependent redistribution of a CaHA-CMC nodule under vibrational energy;

FIG. 7B illustrates the increase in lateral distribution area of the CaHA-CMC nodule following vibrational treatment;

FIG. 7C illustrates the decrease in aspect ratio of the CaHA-CMC nodule during vibrational treatment; and

FIG. 7D illustrates the increase in major axis length of the CaHA-CMC nodule following vibrational treatment.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range. Ranges provided herein are understood to include all individual integer values and all subranges within the ranges.

All references cited herein are hereby incorporated by reference in their entireties. In the event of a conflict in a definition in the present disclosure and that of a cited reference, the present disclosure controls.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care films and/or the oral care compositions of the instant disclosure can be free or essentially free of all components and elements positively recited throughout the instant disclosure. In some instances, the oral care films and/or oral care compositions of the present disclosure may be substantially free of non-incidental amounts of the ingredient(s) or compound(s) described herein. A non-incidental amount of an ingredient or compound is the amount of that ingredient or compound that is added into the oral care films and/or the oral care composition by itself. For example, an oral care film and/or an oral care composition may be substantially free of a non-incidental amount of an ingredient or compound, although such ingredient(s) or compound(s) may be present as part of a raw material that is included as a blend of two or more compounds.

As used herein, the terms “dermal fillers” and “fillers” refer to any composition that may be injected into a subject’s skin (e.g., into a subject’s dermis) to correct, minimize, or otherwise obscure skin defects and/or to fulfill a structural function. These compositions may be used, for example, to create smoother and/or fuller appearance in the face. Exemplary compositions include, but are not limited to, hyaluronic acid; calcium hydroxylapatite (CaHA); polymethyl methacrylate (PMMA); allogenic fat; autologous fat; hydrogel; platelet-rich plasma; platelet-rich fibrin; and mixtures thereof.

As used herein, the terms “dermal filler aggregates” and “aggregates” refer to clumps or clusters of injected dermal fillers that can form beneath a subject’s skin, which may lead to palpable or visible irregularities, lumps, or nodules in the treated area.

One skilled in the art will understand that a subject’s skin includes multiple layers, including the epidermis, which is the outermost layer of skin. The epidermis is understood to have sublayers, including (listed in order from the outermost sublayer to the innermost sublayer), the stratum corneum, stratum lucideum, stratum granulosum, stratum spinosum, and stratum basale. The terms “skin” and “epidermis” may be used interchangeably herein. Accordingly, it is understood that application of a topical serum to a subject’s skin may include application of the serum to the epidermis and delivery of the serum to the stratum corneum.

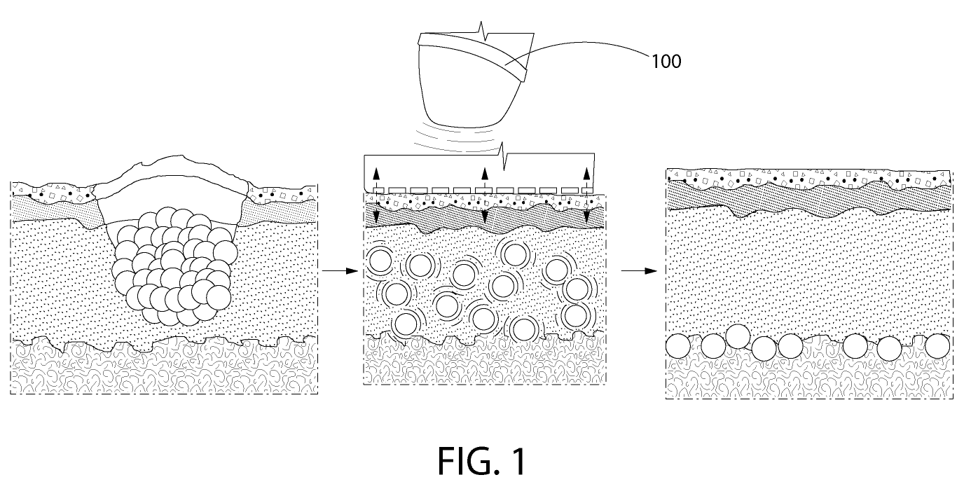

Referring to FIGS. 1-6, embodiments of the present disclosure relate to methods for reducing, removing, or dispersing dermal filler aggregates in a subject through the application of vibrational or mechanical energy to the skin. These methods provide minimally invasive approaches for improving the distribution of dermal fillers within tissue and addressing palpable or visible nodules, clumps, or other irregularities that may form following injection of dermal filler materials.

Dermal filler aggregates may develop due to a variety of factors, including injection technique, product rheology, tissue characteristics, or post-procedural manipulation of the treated area. Such aggregates can result in aesthetic dissatisfaction, patient discomfort, or the need for corrective procedures. Conventional techniques for managing dermal filler irregularities, such as manual massage, needle-based disruption, or enzymatic dissolution, may be inconsistent, uncomfortable, or limited to specific filler chemistries.

The methods described herein utilize controlled application of vibrational energy to promote dispersion or redistribution of filler material within the surrounding tissue. By delivering energy at or near the location of the aggregate, the methods can facilitate breakdown of cohesive structures within the filler, reduce palpability, and improve aesthetic appearance without the need for invasive procedures. In some embodiments, the device is positioned directly over the aggregate or translated across the treatment area in vertical, horizontal, circular, or sweeping motions.

As illustrated schematically in FIG. 1, application of vibrational energy to a region containing a dermal filler aggregate can mechanically disrupt the localized accumulation and promote redistribution of the filler material within surrounding soft tissue. FIG. 1 depicts a representative sequence in which a focal accumulation of filler is present within the tissue, vibrational energy is applied through the device 100, and the aggregate becomes dispersed into a smoother and more uniform distribution.

Without being bound by theory, it is believed that vibrational energy generates localized mechanical waves capable of propagating into the tissue layers where dermal fillers reside. These waves can momentarily decrease cohesive forces within the filler material, allowing the aggregate to deform, elongate, or redistribute within the surrounding tissue planes. This mechanical redistribution can produce both lateral spreading and axial flattening of the aggregate, resulting in a smoother contour without necessitating puncture of the skin or chemical degradation of the filler.

Additional preclinical and clinical observations further support these principles. Studies have demonstrated that vibrational energy can increase the lateral distribution area of CaHA accumulations, reduce aggregate height, and alter aggregate morphology in a time-dependent manner. Clinical reports also show rapid smoothing of early-onset nodules following vibrational energy application, with sustained aesthetic improvement.

In certain embodiments, and without being bound by theory, the mechanical effects of vibrational energy described herein may also facilitate redistribution or clearance of localized accumulations within glandular or ductal structures of the skin or adjacent tissue. For example, in anatomical regions such as the eyelids, vibrational energy applied to the skin surface may assist in relieving blockages associated with glandular structures, including meibomian glands, by promoting mechanical mobilization of obstructive material. Such ancillary effects may occur as a result of the same vibrational mechanisms that facilitate dispersion of dermal filler aggregates, without altering the primary purpose or scope of the methods described herein.

Embodiments of the present methods may be tailored based on the characteristics of the treatment area, including filler type, aggregate size, tissue density, and anatomical location. Optional features such as visible light application, variable vibration patterns, or region-specific motion paths may be employed to optimize comfort or enhance treatment uniformity. The non-invasive nature of the methods enables application across multiple facial or body regions and may allow practitioners to achieve predictable, incremental improvements with minimal downtime.

The following sections describe exemplary devices, components, and treatment methodologies suitable for performing the dermal filler aggregate reduction procedures described herein. These embodiments are provided for illustrative purposes and are not intended to limit the scope of the present disclosure.

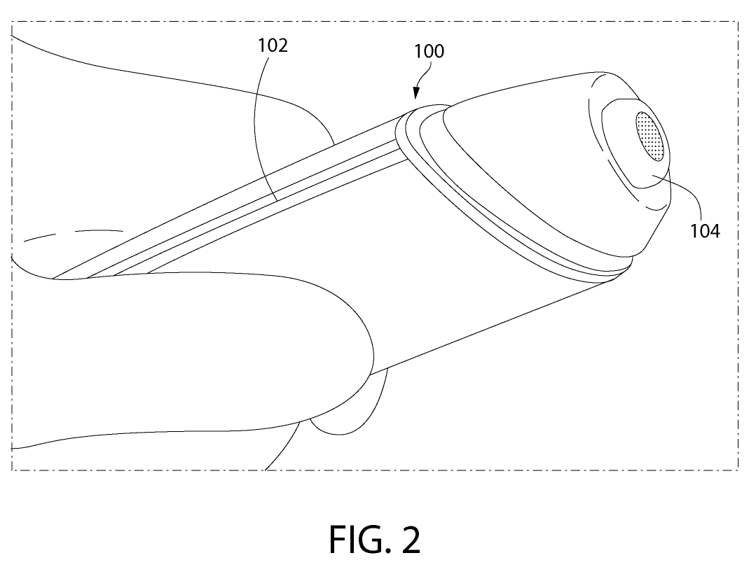

Referring to FIGS. 2-4, embodiments of the present disclosure relate to devices configured to apply vibrational or mechanical energy to a subject’s skin for purposes of reducing, removing, or dispersing dermal filler aggregates. Although specific structural features are illustrated in the accompanying figures, the methods described herein can be performed using any device capable of contacting the skin and delivering vibrational energy, and the present disclosure is not limited to the particular examples shown.

As illustrated in FIG. 2, an exemplary device 100 includes a body 102 and a head 104 that together form a handheld apparatus suitable for contacting a subject’s skin during treatment. The body 102 extends longitudinally and is shaped or contoured to provide an ergonomic grip for a practitioner. The body 102 is hollow or partially hollow to house one or more operational components of the device 100, including a vibration mechanism, control electronics, and/or a power source.

The body 102 has a first end and a second end. The first end forms a terminal end of the device 100, while the second end is configured to couple or removably couple to the head 104. The coupling is achieved through any suitable mechanism, including threading, snap-fit engagement, press-fit engagement, or other connection features that maintain secure attachment during use.

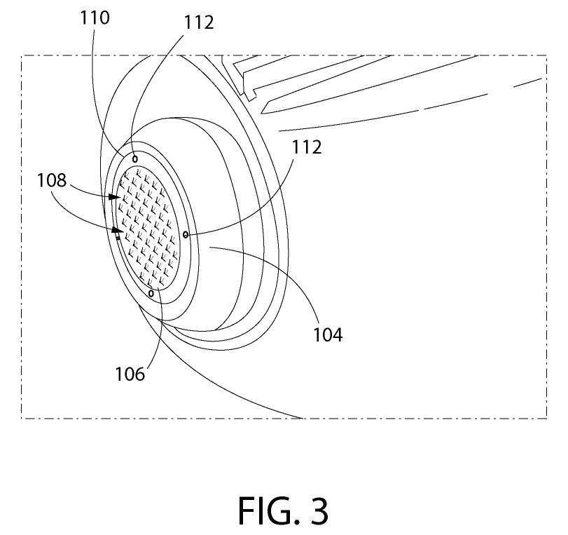

Referring to FIG. 3, the head 104 includes a first end configured for coupling to the body 102 and a second end forming an exterior treatment surface configured to contact a subject’s skin. The treatment surface is planar, substantially planar, curved, convex, or otherwise shaped to enable consistent application of vibrational energy to the target area.

The head 104 includes a contact surface 106 positioned at or near its distal end. In the illustrated embodiment, the contact surface 106 comprises one or more surface features, such as a plurality of tips 108, protrusions, nodes, or other raised structures configured to interact with the skin when the device 100 is actuated. The tips 108 extend perpendicularly or substantially perpendicularly from the contact surface 106 and facilitate distribution of vibrational energy across the skin surface. The tips 108 are configured not to penetrate through the stratum corneum, enabling the dermal filler aggregate reduction methods to be performed in a minimally invasive manner.

The plurality of tips 108 is arranged in an array or pattern on the contact surface 106. The array is continuous or discontinuous and assumes any geometric shape, including squares, rectangles, circles, triangles, rhomboids, trapezoids, or other regular or irregular polygonal configurations. The number, height, spacing, and geometry of the tips 108 vary depending on treatment requirements, provided that the tips are configured to contact—but not penetrate—the skin’s outer surface. In certain embodiments, the tips 108 have a height between about 0.05 mm and about 0.5 mm.

The contact surface 106 and tips 108 are formed from monocrystalline silicon, polymers, metals, ceramics, composites, or other materials suitable for forming durable structures configured for repetitive skin contact. In particular embodiments, the tips 108 are integrally formed with the contact surface 106. In other embodiments, the tips 108 are formed separately and attached to the surface 106.

During use, the head 104 and contact surface 106 are positioned against the subject’s skin such that vibrational energy generated by the device 100 is transmitted directly into the target treatment area. The head 104 can be oriented perpendicular or at an oblique angle relative to the skin surface depending on practitioner preference, treatment location, or the size of the dermal filler aggregate. The configuration of the contact surface 106 and tips 108 enables consistent skin contact during static placement or during motion across the treatment area.

In some embodiments, the contact surface 106 is a substantially flat surface that is devoid of a plurality of tips 108, protrusions, nodes, or other raised structures. In such embodiments, vibrational energy is transmitted to the subject’s skin through direct planar contact between the contact surface 106 and the skin, enabling dispersion of dermal filler aggregates without reliance on surface features extending from the device.

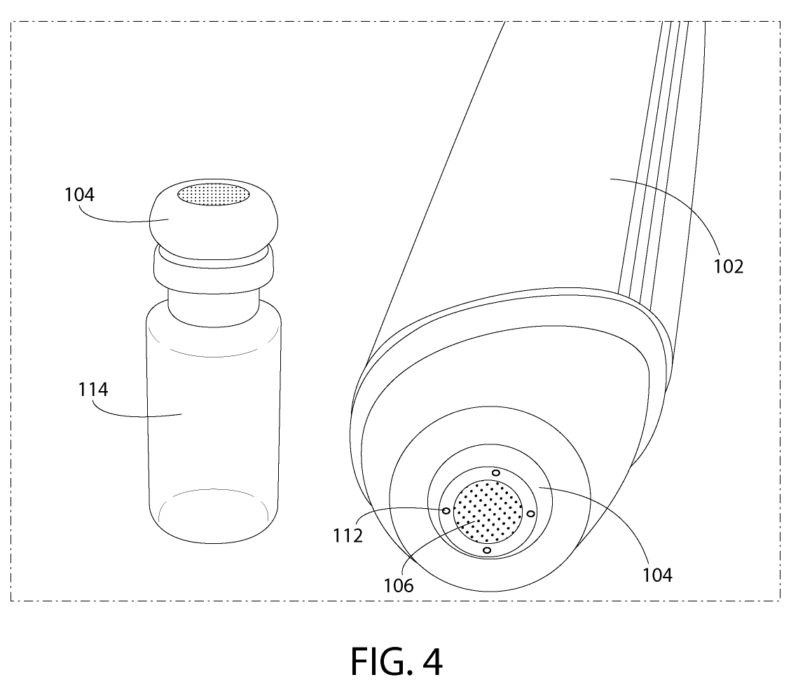

As shown in FIG. 4, the device 100 can include an optional cartridge 114 or internal housing positioned within the body 102. The cartridge 114 is removable or replaceable in certain configurations. Although FIG. 4 depicts a cartridge 114 positioned within the body 102, the presence or absence of the cartridge is not determinative of the dermal filler aggregate reduction methods described herein, which are performed through application of vibrational energy to the skin. Accordingly, the cartridge 114 is not required for the performance of such methods unless expressly stated.

The device 100 can further include an optional visible light source 120 configured to emit red light, blue light, near-infrared light, or combinations thereof. The light source 120 is positioned within the body 102 or head 104 and oriented to direct light toward the skin surface during application of vibrational energy. In certain embodiments, visible light is applied to provide adjunct skin-conditioning effects or enhance user comfort.

The device 100 includes a vibration mechanism configured to generate mechanical oscillations during use. The vibration mechanism comprises one or more of an eccentric rotating mass motor, a linear resonant actuator, a piezoelectric transducer, or another vibration-generating assembly. The device 100 delivers vibrational energy at one or more speeds, frequencies, or amplitudes depending on the treatment protocol. In some embodiments, the device 100 provides vibration speeds of at least 3,000 revolutions per minute (RPM), or speeds from about 3,000 RPM to about 15,000 RPM.

The structural features illustrated in FIGS. 2-4 are provided as examples, and the methods described herein can be performed using a wide variety of devices configured to deliver vibrational energy to the skin. The present disclosure is not limited to the form or configuration of the exemplary device 100 shown in the figures.

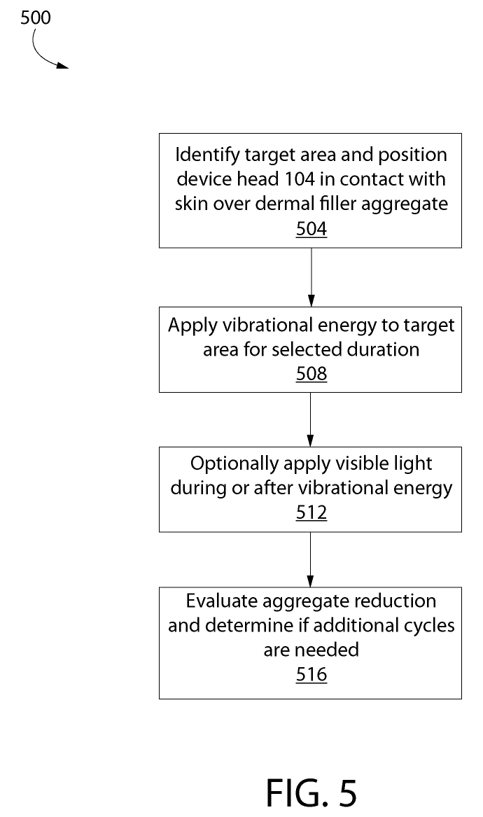

Referring to FIG. 5, an exemplary method 500 for reducing, removing, or dispersing a dermal filler aggregate in a subject will now be described. The method 500 is performed using the device 100 described above or using any other device capable of contacting the skin and delivering vibrational energy to a targeted region. The sequence of steps provided herein is illustrative and can be modified, reordered, or combined as appropriate for a particular treatment protocol.

In step 504, the practitioner identifies a target area of the subject’s skin in which a dermal filler aggregate is present. The aggregate can be detected visually or through palpation, and the practitioner positions the head 104 of the device 100 at or near the location of the aggregate. The head 104 is brought into full contact with the subject’s skin to ensure effective transmission of vibrational energy during treatment.

In step 508, the practitioner applies vibrational energy to the target area by actuating the vibration mechanism of the device 100. Actuation generates mechanical oscillations that propagate through the head 104 and contact surface 106 and into the underlying tissue layers. The vibrational energy is delivered for a selected duration appropriate to facilitate reduction, removal, or dispersion of the dermal filler aggregate. In various embodiments, the vibrational energy is applied for a period from about 5 seconds to about 120 seconds, including periods of about 10 seconds, about 20 seconds, about 30 seconds, about 45 seconds, or about 60 seconds.

In certain embodiments, a topical gliding agent, such as a non-active serum or other lubricating medium, can be applied to the skin surface prior to or during treatment to facilitate smoother movement of the device across the target area and enhance practitioner control. The gliding agent does not participate in chemical modification or dissolution of the dermal filler, but instead serves to optimize mechanical contact during vibrational energy delivery.

During application of vibrational energy, the practitioner can maintain the head 104 in a stationary position directly over the dermal filler aggregate to concentrate energy at the location of the aggregate. Alternatively, or additionally, the practitioner can translate the device 100 across the target area using vertical, horizontal, circular, or sweeping motion patterns. Movement of the device 100 can facilitate distribution of vibrational energy across a wider region, improve blending between the aggregate and surrounding filler material, or address aggregates of irregular shape or size.

In certain embodiments, a topical gliding agent, such as a non-active serum or other lubricating medium, can be applied to the skin surface prior to or during treatment to facilitate smoother movement of the device across the target area and enhance practitioner control. The gliding agent does not participate in chemical modification or dissolution of the dermal filler, but instead serves to optimize mechanical contact during vibrational energy delivery.

The practitioner may also pass the device repeatedly over the aggregate in sequential strokes. In some clinical applications, multiple passes, such as 10 to 20 repetitions, have been observed to promote accelerated smoothing and enhance redistribution of the filler material.

As vibrational energy is delivered, mechanical waves generated by the oscillation of the device 100 can travel into the tissue plane containing the dermal filler. These waves can momentarily disrupt cohesive forces within the filler material, allowing the aggregate to deform, elongate, or redistribute within the surrounding tissue. In certain embodiments, application of vibrational energy reduces the palpability of the aggregate or improves the smoothness or contour of the treated region.

In some embodiments, the method 500 additionally includes applying visible light (step 512) from light source 120 during or after delivery of vibrational energy. Light application including red or blue light can provide ancillary skin-conditioning benefits, enhance patient comfort, or reduce post-treatment redness or irritation. Use of visible light is optional and is not required to achieve dermal filler aggregate reduction.

In step 516, the practitioner evaluates the treated region to assess changes in aggregate size, palpability, or visual appearance. If further dispersion is desired, the practitioner can reapply the device 100 to the same region or to adjacent regions until a satisfactory level of correction is achieved. The practitioner can vary the duration of energy application, the movement pattern, or the applied pressure depending on the response observed in the tissue.

The method 500 can include repeating steps 508–516 for additional treatment cycles if desired. Multiple rounds of vibrational energy application can be administered to achieve incremental improvements in filler distribution or to address larger or more cohesive aggregates. In some embodiments, the method is repeated from about 1 to about 6 times within a single treatment session.

The method 500 can be performed on any region of the body where dermal fillers are commonly injected, including the face, lips, chin, jawline, temples, hands, or other suitable locations. Because the methods described herein are minimally invasive and rely on vibrational energy rather than chemical dissolution or needle-based disruption, they can be compatible with a wide range of filler chemistries, including hyaluronic acid, calcium hydroxyapatite, polymethyl methacrylate, and autologous or allogenic fat.

These methods can be applied to dermal filler aggregates arising immediately or shortly after injection, including early-onset CaHA accumulations, which according to clinical reports may respond rapidly to vibrational redistribution techniques.

The steps described above are provided as examples, and the sequence, duration, and specific parameters of method 500 can be tailored based on the filler material, the location of the aggregate, patient characteristics, and practitioner preference. Embodiments of the present disclosure encompass variations of method 500 that achieve reduction, removal, or dispersion of dermal filler aggregates through application of vibrational energy to the skin.

### Examples

The following Example illustrates an embodiment of the dermal filler aggregate reduction methodology described herein. This Example is provided to aid in understanding the present disclosure and is not intended to limit the scope of the claims. Variations in materials, treatment parameters, or device configurations can be made without departing from the spirit of the present disclosure.

### Example 1: Reduction and Dispersal of Calcium Hydroxyapatite Dermal Filler Aggregates

A synthetic skin model (SimSkin suture pad, approximately 3 in × 3 in × 0.75 in) was used to simulate human dermal tissue. A commercially available calcium hydroxyapatite (CaHA) dermal filler (Radiesse®, Merz Aesthetics, Raleigh, NC) was injected into the synthetic skin model in serial bolus injections to create discrete, palpable aggregates. Each bolus formed a cohesive mass representative of nodules or irregularities that may appear following dermal filler placement in clinical settings.

Vibrational energy was applied to each aggregate using the device 100 for increasing intervals ranging from 10 seconds to 120 seconds. Each bolus received vibrational treatment at a controlled location on the skin surface directly above the aggregate. During each treatment interval, the device 100 was either held stationary over the aggregate or moved in vertical, horizontal, or circular motions to simulate common practitioner techniques. Boluses were evaluated both immediately after treatment and after a brief rest period approximating tissue relaxation.

Following application of vibrational energy, the boluses were assessed using tactile palpation and visual inspection. Palpability was scored on a relative subjective scale from 0 to 5, where 0 indicated minimal or no detectable mass and 5 indicated a firm, cohesive, highly palpable aggregate. Evaluations included assessment of aggregate firmness, spread, surface contour, and resistance to deformation.

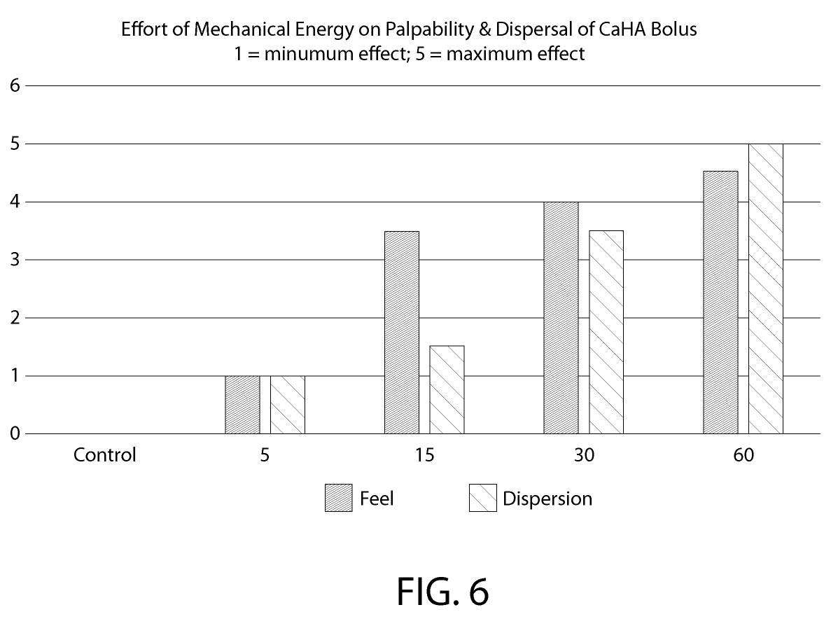

FIG. 6 illustrates the relationship between vibrational energy application time and the corresponding decrease in aggregate palpability for CaHA filler. As shown, aggregates treated for longer durations demonstrated progressively lower palpability scores and greater visible spreading within the simulated tissue. Aggregates treated for 60–120 seconds exhibited marked softening and redistribution relative to untreated controls, which retained their initial cohesiveness and volume.

Qualitative observations during treatment indicated that shorter treatment durations (10–20 seconds) produced minor softening, while intermediate durations (30–60 seconds) resulted in noticeable deformation and elongation of the bolus. Longer durations (90–120 seconds) further enhanced aggregate dispersal, producing smoother contours and substantial reduction in nodularity. When sweeping motions were applied, the filler demonstrated increased lateral spread compared to stationary-only treatments.

Additional treatment rounds were applied to several boluses to evaluate cumulative effects. Repeated applications of vibrational energy (e.g., two or three cycles of 30–60 seconds each) produced additive improvements in dispersal and reduction of aggregate firmness, supporting the utility of multi-stage treatment approaches in certain cases.

Together, the results demonstrate that application of vibrational energy facilitates mechanical disruption and redistribution of CaHA dermal filler aggregates. Increased treatment duration and repeated application cycles correlated with enhanced dispersal. These findings support the use of the methods described herein for improving the aesthetic appearance of dermal filler irregularities and reducing palpable nodules across a range of filler types and anatomical treatment areas.

It should be understood that this Example is illustrative and that variations in filler type, aggregate size, device parameters, or evaluation methodology can be employed without departing from the scope of the present disclosure. Additional embodiments and experimental configurations will be apparent in light of the present teachings.

### Example 2: Hydrogel Model Demonstrating Vibrational Dispersion of Filler Accumulations

A skin-mimicking hydrogel model was used to further evaluate the effects of vibrational energy on CaHA-CMC accumulations. Undiluted CaHA-CMC (0.2 mL) was injected into the hydrogel to form a discrete, cohesive nodule. Vibrational energy was applied to the nodule using the device 100 operated at approximately 8800 RPM for 30 seconds.

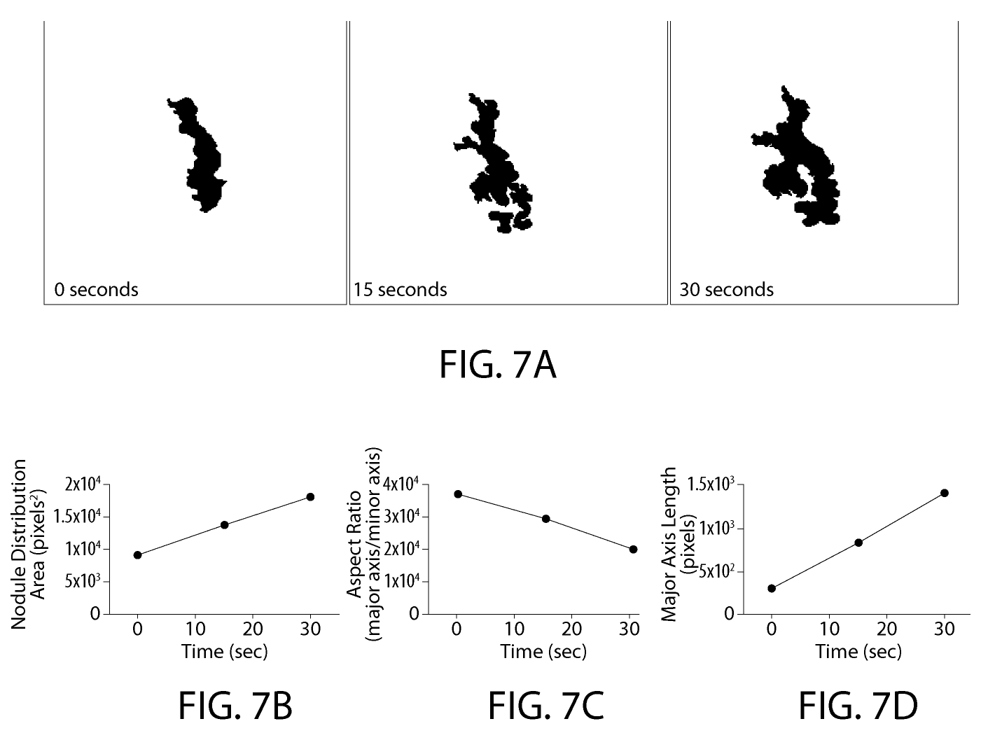

As shown in FIG. 7A, macroscopic images of the hydrogel before treatment and at 15 seconds and 30 seconds after vibration exposure demonstrate progressive redistribution of the CaHA-CMC nodule. The nodule gradually spreads laterally within the hydrogel matrix, producing a visibly flatter and broader profile with increasing treatment duration. FIG. 7A visually highlights the time-dependent morphological changes induced by vibrational energy.

FIG. 7B illustrates quantitative increases in the lateral distribution area of the nodule following vibration exposure. The measured area approximately doubled after 30 seconds of treatment, reflecting pronounced lateral dispersion of CaHA-CMC particles within the constrained medium. Increased lateral area is indicative of effective mechanical spreading of the aggregate.

FIG. 7C shows a decrease in the nodule’s aspect ratio (major axis/minor axis) over the same time interval. A decreasing aspect ratio corresponds to a shift from an elongated shape to a more isotropic, rounded morphology. This transformation is consistent with vibrationally induced redistribution that reduces localized height while promoting uniform lateral spreading.

FIG. 7D depicts a substantial increase in the major axis length of the nodule after vibrational application. This elongation confirms that vibrational energy produces horizontal expansion of the CaHA-CMC bolus while concurrently reducing its vertical projection. Together with the data from FIGS. 7B and 7C, FIG. 7D demonstrates coordinated morphological shifts characteristic of mechanical dispersion.

Collectively, the findings shown in FIGS. 7A-7D support the conclusion that vibrational energy facilitates mechanical deformation and redistribution of CaHA particles within a constrained medium. The time-dependent lateral spreading, reduced aspect ratio, and increased major axis length correlate strongly with the mechanisms described herein for smoothing and dispersing dermal filler aggregates in clinical applications.

### Example 3: Clinical Reduction of an Early-Onset CaHA Dermal Filler Nodule

A 61-year-old male patient presented with an early-onset, non-tender CaHA-CMC nodule on the dorsum of the hand approximately three hours after injection. The nodule measured approximately 0.2 mL and was visibly and palpably detectable.

Vibrational energy was applied to the treatment area for approximately two minutes using repeated passes of the device across the nodule. No saline infiltration or enzymatic agents were used. A topical, non-active serum was applied as a gliding agent to facilitate smooth device movement.

Immediately after treatment, the nodule size was reduced by approximately 95%, with marked smoothing of the skin surface. Mild, transient erythema was observed immediately following treatment.

At a 24-hour follow-up assessment, the nodule was no longer visible or palpable. The patient reported no recurrence of nodularity at a four-week follow-up and noted improved contour and skin quality in the treated region.

This clinical case demonstrates that vibrational energy can rapidly and substantially disperse early-onset CaHA accumulations and supports the practical applicability of the methods described herein.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A method for reducing, removing, or dispersing a dermal filler aggregate in a subject, the method comprising:
(a) contacting a target area of the subject’s skin having a dermal filler aggregate with a device, the device comprising a head and being configured to apply vibrational energy to the target area; and
(b) applying vibrational energy to the target area via the device for a duration sufficient to facilitate reduction, removal, or dispersion of the dermal filler aggregate.

2. The method of claim 1, wherein the vibrational energy comprises a vibration speed of at least 3,000 revolutions per minute (RPM).

3. The method of claim 1, wherein the vibrational energy comprises a vibration speed of from about 3,000 RPM to about 15,000 RPM.

4. The method of claim 1, wherein the vibrational energy comprises a vibration speed of from about 5,800 RPM to about 8,800 RPM.

5. The method of claim 1, wherein applying vibrational energy comprises transmitting vibrational waves proximally to a location of the dermal filler aggregate to cause the aggregate and/or adjacent tissue to vibrate.

6. The method of claim 1, wherein the dermal filler comprises at least one material selected from the group consisting of hyaluronic acid, calcium hydroxyapatite, polymethyl methacrylate (PMMA), allogenic fat, autologous fat, hydrogel, platelet-rich plasma, platelet-rich fibrin, and combinations thereof.

7. The method of claim 6, wherein the dermal filler comprises hyaluronic acid.

8. The method of claim 6, wherein the dermal filler comprises calcium hydroxyapatite.

9. The method of claim 1, wherein contacting the subject’s skin comprises moving the device across the target area in a pattern comprising vertical, horizontal, circular, or sweeping motions.

10. The method of claim 1, wherein the device is held substantially stationary at the location of the dermal filler aggregate during at least a portion of applying the vibrational energy.

11. The method of claim 1, wherein applying vibrational energy comprises contacting the target area for from about 5 seconds to about 120 seconds.

12. The method of claim 1, wherein the method is repeated from 1 to 6 times to achieve the desired reduction, removal, or dispersion of the dermal filler aggregate.

13. The method of claim 1, wherein contacting the subject’s skin comprises contacting the skin with a plurality of tips extending from the device.

14. The method of claim 13, wherein the plurality of tips have a height of between 0.05 mm and 0.5 mm.

15. The method of claim 13, wherein the plurality of tips are configured not to penetrate through the stratum corneum.

16. The method of claim 1, further comprising applying light from a visible light source to the target area contemporaneously with applying the vibrational energy.

17. The method of claim 16, wherein the light is red light or blue light.

18. The method of claim 17, wherein the light comprises red light having a wavelength from about 620 nm to about 700 nm.

19. The method of claim 1, wherein applying vibrational energy reduces palpability of the dermal filler aggregate.

20. The method of claim 1, wherein applying vibrational energy improves the dispersion profile of the dermal filler within the treated area.
